(19) United States
(12) Patent Application Publication
(10) Pub. No.: US 2025/0206796 A1
(43) Pub. Date: Jun. 26, 2025

(54) USE OF VIRAL IL-6 IN CANCER THERAPY

(71) Applicant: The Regents of the University of California, Oakland, CA (US)

(72) Inventors: Michiko Shimoda, Oakland, CA (US); Yoshihiro Izumiya, Oakland, CA (US)

(21) Appl. No.: 18/852,101

(22) PCT Filed: Apr. 12, 2023
(86) PCT No.: PCT/US2023/018379

§ 371 Date: Sep. 27, 2024

Related U.S. Application Data
(60) Provisional application No. 63/330,532, filed on Apr. 13, 2022.

(51) Int. Cl. C07K 14/54; A61K 38/00; C07K 14/005

(52) U.S. Cl. CPC C07K 14/5412; C07K 14/005; A61K 38/00; C12N 2710/16422

(57) ABSTRACT

The present invention resides in the discovery that the viral homolog of human interleukin 6 (vIL-6) originated from Kaposi's sarcoma herpesvirus (KSHV), unlike human IL-6, does not trigger negative regulatory mechanisms during an inflammatory response and therefore can be used in cancer treatment to support an unhindered anti-cancer immunity. Accordingly, this invention provides a novel method for cancer therapy by using vIL-6 to increase the immune-stimulating properties of dendritic cells, during dendritic cell production from monocytes in preparation for dendritic cell transfer therapy, as well as by co-administering vIL-6 with an anticancer therapeutic agent to achieved enhanced therapeutic immunological effects. A composition and kit for such use are also described.

### RELATED APPLICATIONS

This application is a U.S. National Stage of PCT/US2023/018379, International Filing Date Apr. 12, 2023, which claims priority to U.S. Provisional Patent Application No. 63/330,532, filed Apr. 13, 2022, the contents of which are hereby incorporated in the entirety for all purposes.

### SEQUENCE LISTING

The contents of the electronic sequence listing (081906-1379565-248310PC_ST26.xml; Size: 16,313 bytes; and Date of Creation: Sep. 16, 2024) is herein incorporated by reference in its entirety.

### BACKGROUND OF THE INVENTION

Viral IL-6 is a cytokine (˜28 kD) encoded by Kaposi's sarcoma herpesvirus (KSHV) and is considered to be a human interleukin-6 (hIL-6) homolog, triggering JAK/STAT pathways for cellular proliferation and survival. Previous studies have shown that hIL-6 triggers anti-inflammatory negative feedback mechanisms, blocks dendritic cell differentiation, and induces macrophage differentiation instead. It has now been discovered that, unlike hIL-6, vIL-6 does not trigger negative feedback mechanisms, does not prevent dendritic cell differentiation, and rather promotes dendritic cell differentiation and M1 macrophage differentiation from monocytes. As such, vIL-6 can be used as an adjuvant in combination with current cancer therapeutic regimen to promote anti-tumor immunity.

### BRIEF SUMMARY OF THE INVENTION

This invention relates to a virally derived homolog of interleukin-6 (vIL-6), which, like IL-6, promotes inflammatory responses but, unlike IL-6, does not trigger negative feedback regulation to reduce the inflammatory response. This feature provides a unique benefit in the usage of vIL-6 in that a therapeutically desirable immune response is not limited by self-downregulation. Thus, in the first aspect of this invention, a method is provided for an enhanced cancer therapy where vIL-6 is administered in combination with an anti-cancer therapeutic agent, especially an anti-cancer immunotherapeutic agent. The use of vIL-6 and this property of vIL-6 to increase the inflammatory response in a scenario where this property may be desirable such as cancer immunotherapy by administering to a patient in need thereof an effective amount of vIL-6, either in protein form or in nucleic acid form, such as RNA- or DNA-based delivery (e.g., viral vector-based delivery) of a vIL-6-encoding polynucleotide sequence. Alternatively, vIL-6 can be added to cell culture as a supplement to enhance the recovery and the efficacy of dendritic cells to eradicate cancer cells for dendritic cell transfer therapy.

In some embodiments, the vIL-6 administered is a vIL6 protein. In some embodiments, the vIL-6 administered is a nucleic acid comprising a polynucleotide sequence encoding the vIL-6 protein. In some embodiments, the nucleic acid is RNA. In some embodiments, the nucleic acid is DNA, including a DNA-based expression cassette, encoding the vIL-6 protein. In some embodiments, the nucleic acid is a viral vector encoding the vIL-6 protein.

In some embodiments, the anti-cancer therapeutic agent comprises an anti-cancer immunotherapeutic agent. In some embodiments, the administering step comprises systemic or local administration of at least one anti-cancer agent and vIL-6, e.g., by oral ingestion or nasal inhalation, topical contact, administration as a suppository, intravenous, intraperitoneal, intramuscular, intralesional, intratumoral, intrathecal, intranasal, intraosseous, or subcutaneous administration to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arterial, intradermal, subcutaneous, intraperitoneal, intraventricular, intraosseous, and intracranial. Other modes of delivery include, but are not limited to, the use of liposomal formulations, intravenous infusion, transdermal patches, etc.

In some embodiments, anti-cancer therapeutic agents or therapies that can be used in connection with vIL-6 of the present disclosure can include but are not limited to therapeutic antibodies comprising a specificity for an immune checkpoint molecule such as PD1, PD-L1, LAG3, CTLA-4, A2AR, TIM-3, BTLA, CD276, CD328, VTCN1, IDO, KIR, NOX2, VISTA, OX40, CD27, CD28, CD40, CD122, CD137, GITR, ICOS. Exemplary antibodies comprising a specificity for an immune checkpoint molecule include, but are not limited to an anti-PD1 antibody selected from dostarlimab, pembrolizumab, nivolumab, and pidilizumab.

In some embodiments, the anti-cancer therapeutic agents or therapies that can be used in connection with vIL-6 in accordance with the present disclosure can include but are not limited to therapeutic cytokines and/or cytokine fusion proteins such as IL-2, IL-2 muteins, and IL-15/IL-15RA complex, as well as cancer vaccines including mRNA-based cancer vaccines and adjuvants such as talimogene laherparepvec (T-VEC), and cancer cell therapies including adoptive cell therapy (tumor-infiltrating lymphocyte therapy, engineered T cell receptor therapy, chimeric antigen receptor T cell therapy, and natural killer cell therapy) and ex vivo preparation of dendritic cells for dendritic cell-based transfer therapy.

The second aspect of the present invention provides a composition for treating cancer, especially for improved or enhanced therapeutic efficacy. The composition comprises (1) vIL-6; (2) an anti-cancer therapeutic agent (e.g., an anti-cancer agent for immunotherapy); and (3) a physiologically acceptable excipient. In some embodiments, the vIL-6 is a vIL-6 protein. In some embodiments, the vIL-6 is a nucleic acid comprising a polynucleotide sequence encoding the vIL-6 protein. In some embodiments, the nucleic acid is DNA or RNA. In some embodiments, the nucleic acid is a viral vector, such as an adenovirus vector or adenovirus-associated viral vector.

In a third aspect, the present invention provides a kit for treating cancer especially for cancer immunotherapy with improved or enhance therapeutic efficacy. The kit includes a first container containing a first composition comprising an effective amount of vIL-6 as described above or herein and a second container containing a second composition comprising an anti-cancer therapeutic agent (e.g., an anti-cancer agent for immunotherapy). In some embodiments, the vIL-6 is a vIL-6 protein. In some embodiments, the vIL-6 is a nucleic acid comprising a polynucleotide sequence encoding the vIL-6 protein. In some embodiments, the nucleic acid is DNA or RNA. In some embodiments, the nucleic acid is a viral vector, such as an adenoviral vector or adenovirus-associated viral vector. Optionally, the kit further includes user instruction material providing description of dosing arrangements and its intended use.

In a fourth aspect, the present invention provides for a vIL-6-containing supplement that can be used during the process of producing dendritic cells from blood-derived monocytes and/or during the manipulation of such dendritic cells or other dendritic cells from other sources such as dendritic cells isolated from patients' blood, to increase their immune-response-stimulating properties. During this process, monocytes or dendritic cells are first extracted from a cancer patient's body (e.g., blood) and then incubated ex vivo in a cell culture medium containing a vIL-6 protein or nucleic acid encoding the vIL-6 protein, the dendritic cells optionally also being exposed to one or more antigens (e.g., peptide antigens) derived from the specific cancer the patient suffers from. After ex vivo culture, these dendritic cells would then be administered back into the patient's body for the purpose of eliminating the cancer cells. In some embodiments, the vIL-6 is a vIL-6 protein. In some embodiments, the vIL-6 is a nucleic acid comprising a polynucleotide sequence encoding the vIL-6 protein. In some embodiments, the nucleic acid is DNA. In some embodiments, the nucleic acid is RNA. In some embodiments, the nucleic acid is an expression cassette capable of directing the expression of a vIL-6 protein, for example, a viral vector (such as an adenoviral vector or adenovirus-associated viral vector) encoding the vIL-6 protein.

### BRIEF DESCRIPTION OF THE DRAWINGS

FIGS. 1A-1C Preparation of biologically active vIL-6. (A) Recombinant vIL-6 protein. Recombinant KSHV vIL-6 protein was expressed with recombinant baculovirus in Sf9 cells and purified with Flag agarose beads. Coomassie staining gel is shown. Marker is depicted on the left. (B) Cell signaling activation. STAT1 activation was probed with phospho-STAT antibodies (Cell Signaling). IFNα, TNFα, and human IL-6 (hIL-6) were used as positive controls for signal activation. Two cell lines were used to consider cell line specific artifacts. (C) Inflammatory gene expression. THP-1 cells were left untreated or treated with vIL-6 (200 ng/ml) for 6 hours, and gene expression was measured by qRT-PCR after normalizing for β-actin.

FIGS. 2A-2C Long term vIL-6 treatment induces inflammatory M1 phenotype in monocytic cells. (A) Long-term vIL-6 treatment of THP-1 cells. THP-1 cells were left untreated or treated with vIL-6 (200 ng/ml) every day for 2 weeks followed by no treatment for 24 hrs, and gene expression was measured by qRT-PCR after normalizing for beta-actin. *p<0.05, **p<0.01. (B and C) Long-term vIL-6 treatment of human primary monocytes. Human primary monocytes were isolated from peripheral blood mononuclear cells (PBMC) by negative selection of magnetic beads. Monocytes (5×10\hat{ }6/ml, 400 uL in 48 well plate, 10% FBS RPMI1640) were left untreated (PBS control) or treated with vIL-6 (100 ng/ml) for 1-2 weeks to allow differentiation of macrophages. (B) representative FACS plots of live monocytes one week after vIL-6 treatment and the average frequencies of live cells and cell irregularity based on FSC and SSC values are shown (n=3). (C) Representative histogram overlays of live macrophages for M2 markers (CD163, CD206, CD11b, CD16) and M1 markers (HLA-DR, CD86) are shown.

FIGS. 3A-3F vIL-6 promotes dendritic cell differentiation while reducing macrophage differentiation. (A) Representative cellular mass images after monocytes cultured under dendritic cell differentiation. Monocytes were isolated from PBMC of 6 healthy donors by negative selection of magnetic beads, and then cultured in the presence of GM-CSF and IL-4 (2×10⁶/ml, 200 uL in 96 U-bottom well plate, 10% FBS RPMI1640, 50 ng/ml GM-CSF, 50 ng/ml IL-4) with PBS (control), vIL-6 (100 ng/ml), or hIL-6 (100 ng/ml) for 1-2 weeks to allow dendritic cell differentiation. (B) Cell number and (C) cell viability of monocytes cultured with hIL-6 or vIL-6. Fold changes over control (PBS) cultures are shown. (D) Representative flow cytometry profiles of monocyte cultures treated with PBS, hIL-6, or vIL-6 under the dendritic cell differentiation condition as described in (A). The frequency of dendritic cells (red gate) and macrophages (blue gate) were analyzed by flow cytometry based on the expression of CD1c and CD11b. (E) The frequency of dendritic cells and that of macrophages as determined in (D) are compared between cultures with PBS, hIL-6, or vIL-6. n=3. ****p<0.0001. (F) The mean fluorescent intensity of inhibitory markers PD-L1 and CD16 on monocytes are analyzed by flow cytometry and compared between cultures with hIL-6, or vIL-6. n=6. *p<0.05, **p<0.01.

FIG. 4 vIL6 amino acid sequence used in this study. (A) vIL-6 amino acid sequence, which is based on the KSHV K2 open reading frame (ORF), with added GS flexible linker to prevent Flag tag from disrupting vIL-6 function (underlined) and Flag epitope tag (bold italic) to allow affinity purification of protein. Signal sequence (22 amino acids) is underlined in italic. (B) Recombinant vIL-6 DNA sequence with optimized codon usage for improved expression in insect cells.

FIGS. 5A-5C Preparation of biologically active vIL-6 in human cell lines for cGMP production purposes. (A) Recombinant vIL-6 protein. Recombinant KSHV wild type vIL-6 protein (vIL-6-Wt-Flag) along with a loss-of-function vIL-6 C/A mutant protein (vIL-6-C/A-Flag) as a control for signal transduction profiling were expressed in human 293FT cell lines and purified with Flag agarose beads. Coomassie staining gel is shown. Marker is depicted on the left. (B) Amino acid sequence (including signal sequence) comparison of vIL-6-Wt and vIL-6 C/A mutant indicating the C/A mutation sites. (C) Cell signaling activation. vIL-6-Wt-Flag and vIL-6-C/A-Flag proteins were produced by insect Sf9 cell line (a) as a control or by human 293FT cell line (b) and used for the assays. In (a), two human cell lines (iSLK and THP-1) were used to consider cell line specific artifacts. STAT3 activation was probed with phospho-STAT antibodies (Cell Signaling).

FIGS. 6A-6D Gene expression analysis of human THP-1 cell line after vIL-6 stimulation. (A) Experimental design. To study a long-term impact of vIL-6 in the growth and functional changes in monocytes, human monocytic cell line THP-1 was treated in various conditions with vIL-6 and TGFβ, as an irrelevant stimulus control (Experiment 1-9). Briefly, THP-1 cells were primed without or with vIL-6 for 2 weeks, rested for 1 day without vIL-6, and treated with vIL-6 or TGFβ for 30 min before RNA-seq analysis and transcription profiling was conducted in (B, C, and D). (B)(i) Principal component analysis of experiment 1-9 in (A). n=3. (ii) Heatmap shows differential transcriptional profiles of THP-1 cells either long vIL-6-primed or vIL-6 short-treated. (C) Venn diagrams to demonstrate the impact of vIL-6 priming on gene expressions. THP-1 cells were primed without (THP-1 in red circle) or with vIL-6 (vIL-6/THP-1 in green circle) followed by stimulation with (a) vIL-6 or (b) TGFβ as a control. The numbers of genes differentially and commonly expressed in each circle are shown. (D) Gene Set Enrichment Analysis (GSEA) for vIL-6 targets. GSEA was performed with the group of genes that are differentially (red or green) or commonly (yellow beige) expressed by THP-1 cells without or with vIL-6 priming as in FIG. 5C (a).

### DEFINITIONS

As used herein, “vIL-6” refers to a viral homolog of the human interleukin-6 (IL-6) molecule. In this application, “vIL-6” encompasses any variant that (1) shares at least 90% sequence identity to the amino acid sequence of an exemplary vIL-6 derived from KSHV ORF K2 (e.g., shown below as SEQ ID NO:1); and (2) shares at least one or more of the functionalities of exemplary vIL-6 of SEQ ID NO:1 such as the capability of binding to the gp130 subunit of the IL-6 receptor to activate the JAK/STAT pathway and/or the AKT pathway. A “vIL-6” protein may be any protein having the requisite amino acid sequence identity (at least 90, 91, 92, 93, 94, 95, 96, 97, 98, or 99%) to the amino acid sequence set forth in SEQ ID NO:1 below but potentially with different post-translational modifications including but not limited to glycosylation, PEGylation, crosslinking, etc. or the presence of one or more non-naturally occurring amino acids such as D-amino acids. In addition, a vIL-6 protein may optionally include one or more additional amino acid sequence (e.g., a signal sequence at the N-terminus), such as one or more heterologous amino acid sequences of at least 2, 3, 4, 5, 6, 7, 8, 9, 10, 11, 12, 13, 14, 15, 16, 17, 18, 19, 20, 25, 30, 35, 40, 45, or up to about 50, about 60, or about 70 amino acids at C- and/or N-terminus of the vIL-6 sequence (such as SEQ ID NO:1). Such heterologous peptide sequences can be of a varying nature, for example, any one of the “tags” known and used in the field of recombinant proteins: a peptide tag such as an AviTag, a peptide allowing biotinylation by the enzyme BirA and so the protein can be isolated by streptavidin, a Calmodulin-tag, a peptide bound by the protein calmodulin, a polyglutamate tag, a peptide binding efficiently to anion-exchange resin such as Mono-Q, an E-tag, a peptide recognized by an antibody, a FLAG-tag, a peptide recognized by an antibody, an HA-tag, a peptide recognized by an antibody, a His-tag, 5-10 histidines bound by a nickel or cobalt chelate, a Myc-tag, a short peptide recognized by an antibody, an S-tag, an SBP-tag, a peptide that specifically binds to streptavidin, a Softag 1 for mammalian expression, a Softag 3 for prokaryotic expression, a Strep-tag, a peptide that binds to streptavidin or the modified streptavidin called streptactin (Strep-tag II), a TC tag, a tetracysteine tag that is recognized by FlAsH and ReAsH biarsenical compounds, a V5 tag, a peptide recognized by an antibody, a VSV-tag, a peptide recognized by an antibody, an Xpress tag; or a covalent peptide tags such as an Isopeptag, a peptide that binds covalently to pilin-C protein, a SpyTag, a peptide that binds covalently to SpyCatcher protein; or a protein tag such as a BCCP tag (Biotin Carboxyl Carrier Protein), a protein domain biotinylated by BirA enabling recognition by streptavidin, a Glutathione-S-transferase (GST) tag, a protein that binds to immobilized glutathione, a Green fluorescent protein (GFP) tag, a protein that is spontaneously fluorescent and can be bound by nanobodies, a Maltose binding protein (MBP) tag, a protein that binds to amylose agarose, a Nus-tag, a Thioredoxin-tag, an Fc-tag, derived from immunoglobulin Fc domain, allow dimerization and solubilization. A tag that can be used for purification on Protein-A Sepharose; as well as other types of tags such as the Ty tag. An exemplary vIL-6 protein has the amino acid sequence set forth below (SEQ ID NO:1):

KLPDAPEFEKDLLIQRLNWMLWVIDECFRDLCYRTGICKGILEPAAIFHL
KLPAINDTDHCGLIGFNETSCLKKLADGFFEFEVLEKFLTTEFGKSVINV
DVMELLTKTLGWDIQEELNKLTKTHYSPPKFDRGLLGRLQGLKYWVRHFA
SFYVLSAMEKFAGQAVRVLDSIPDVTPDVHDK

“Inflammation” refers to an organism's immune response to irritation, toxic substances, pathogens, or other stimuli. The response can involve innate immune components and/or adaptive immunity. Inflammation is generally characterized as either chronic or acute. Acute inflammation is characterized by redness, pain, heat, swelling, and/or loss of function due to infiltration of plasma proteins and leukocytes to the affected area. Chronic inflammation is characterized by persistent inflammation, tissue destruction, and attempts at repair. Monocytes, macrophages, plasma B cells, and other lymphocytes are recruited to the affected area, and angiogenesis and fibrosis occur, often leading to scar tissue.

As used herein, the term “cancer” encompasses various malignant neoplasms characterized by the proliferation of anaplastic cells that tend to invade surrounding tissue and metastasize to new body sites. Non-limiting examples of different types of cancer suitable for treatment using the compositions and methods of the present invention include colorectal cancer, colon cancer, anal cancer, liver cancer, ovarian cancer, breast cancer, lung cancer, bladder cancer, thyroid cancer, pleural cancer, pancreatic cancer, cervical cancer, prostate cancer, testicular cancer, bile duct cancer, gastrointestinal carcinoid tumors, esophageal cancer, gall bladder cancer, rectal cancer, appendix cancer, small intestine cancer, stomach (gastric) cancer, renal cancer (e.g., renal cell carcinoma), cancer of the central nervous system, skin cancer, oral squamous cell carcinoma, choriocarcinomas, head and neck cancers, bone cancer, osteogenic sarcomas, fibrosarcoma, neuroblastoma, glioma, melanoma, leukemia (e.g., acute lymphocytic leukemia, chronic lymphocytic leukemia, acute myelogenous leukemia, chronic myelogenous leukemia, or hairy cell leukemia), lymphoma (e.g., non-Hodgkin's lymphoma, Hodgkin's lymphoma, B-cell lymphoma, or Burkitt's lymphoma), and multiple myeloma.

The term “immunotherapy” refers to any treatment that uses certain parts of a patient's immune system to fight diseases such as cancer. The patient's own immune system is stimulated (or suppressed), with administration of one or more agents for that purpose. In some cases, the immunotherapy is “targeted” or cancer cell-specific. In other cases, immunotherapy can be “untargeted,” which refers to administration of agents that do not selectively interact with immune system cells, yet modulates immune system function. Representative examples of untargeted therapies include, without limitation, chemotherapy, gene therapy, and radiation therapy.

Immunotherapy is one form of targeted therapy that may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). For example, anti-VEGF and mTOR inhibitors are known to be effective in treating renal cell carcinoma. Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

Another example of cancer immunotherapy is the so-called “dendritic cell transfer therapy.” During this process, dendritic cells are either extracted from a patient diagnosed with a specific cancer or generated by ex vivo culture from monocytes taken from the patient. These dendritic cells are then cultured ex vivo for a suitable length of time (for example, up to 12, 18, or 24 hours, overnight, or 12 to 18 or 24 hours) with one or more antigens specific for the cancer the patient suffers from before the “pulsed” dendritic cells are re-introduced into the patient's body, typically by injection (e.g., intravenous injection), to trigger a cancer antigen-specific immune response mediated by primed effector T cells. In the context of the present invention, vIL-6 protein or nucleic acid may be used during the ex vivo culturing process of the dendritic cells extracted from the cancer patient's body or induced from monocytes taken from the patient (e.g., blood), for instance, as an added ingredient in the cell culture medium, to increase the number of the dendritic cells, enhance the cells' vitality, and/or otherwise improve the cells' capability to induce the cancer antigen-specific immune response.

The term “immunogenic chemotherapy” refers to any chemotherapy that has been demonstrated to induce immunogenic cell death, a state that is detectable by the release of one or more damage-associated molecular pattern (DAMP) molecules, including, but not limited to, calreticulin, ATP and HMGB1 (see, e.g., Kroemer et al. (2013), Annu. Rev. Immunol., 31:51-72). In addition, the term “immunogenic chemotherapy” further refers to any chemotherapy that results in priming the immune system such that it leads to enhanced immune activity towards cancer. Specific representative examples of consensus immunogenic chemotherapies include 5′-fluorouracil, anthracyclines, such as doxorubicin, and the platinum drug, oxaliplatin, among others.

“Immune checkpoint” molecules generally encompass a group of molecules on the cell surface of CD4⁺ and/or CD8⁺ T cells that fine-tune immune responses by down-modulating or inhibiting an anti-tumor immune response. Immune checkpoint proteins are well-known in the art and include, without limitation, CTLA-4, PD-1, VISTA, B7-H2, B7-H3, PD-L1, B7-H4, B7-H6, ICO S, HVEM, PD-L2, CD160, gp49B, PIR-B, KIR family receptors, TIM-1, TIM-3, TIM-4, LAG-3, GITR, 4-IBB, OX-40, BTLA, SIRP, CD47, CD48, 2B4 (CD244), B7.1, B7.2, ILT-2, ILT-4, TIGIT, HHLA2, butyrophilins, IDO, CD39, CD73 and A2aR (see, for example, WO 2012/177624).

The term “nucleic acid” or “polynucleotide” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); and Cassol et al., (1992); Rossolini et al., Mol. Cell. Probes, 8:91-98 (1994)). The terms nucleic acid and polynucleotide are used interchangeably with gene, cDNA, and mRNA encoded by a gene.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full length proteins (i.e., antigens), wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

An “antibody” refers to a polypeptide substantially encoded by an immunoglobulin gene or immunoglobulin genes, or fragments thereof, which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially an Fab with part of the hinge region (see, Paul (Ed.) Fundamental Immunology, Third Edition, Raven Press, NY (1993)). While various antibody fragments are defined in terms of the digestion of an intact antibody, one of skill will appreciate that such fragments may be synthesized de novo either chemically or by utilizing recombinant DNA methodology.

Further modification of antibodies by recombinant technologies is also well known in the art. For instance, chimeric antibodies combine the antigen binding regions (variable regions) of an antibody from one animal with the constant regions of an antibody from another animal. Generally, the antigen binding regions are derived from a non-human animal, while the constant regions are drawn from human antibodies. The presence of the human constant regions reduces the likelihood that the antibody will be rejected as foreign by a human recipient. On the other hand, “humanized” antibodies combine an even smaller portion of the non-human antibody with human components. Generally, a humanized antibody comprises the hypervariable regions, or complementarity determining regions (CDR), of a non-human antibody grafted onto the appropriate framework regions of a human antibody. Antigen binding sites may be wild type or modified by one or more amino acid substitutions, e.g., modified to resemble human immunoglobulin more closely. Both chimeric and humanized antibodies are made using recombinant techniques, which are well-known in the art (see. e.g., Jones et al. (1986) Nature 321:522-525).

Thus, the term “antibody,” as used herein, also includes antibody fragments either produced by the modification of whole antibodies or antibodies synthesized de novo using recombinant DNA methodologies (e.g., single chain Fv, a chimeric or humanized antibody). One example is the so-called “nanobody” or single-domain antibody (sdAb), an antibody fragment consisting of a single monomeric variable antibody domain, especially a heavy chain variable domain. Like a whole antibody, a nanobody is able to bind selectively to a specific antigen. With a molecular weight of only 12-15 kDa, nanobodies are much smaller than common antibodies (150-160 kDa) having two heavy chains and two light chains, and even smaller than Fab fragments (˜50 kDa) and single-chain variable fragments (˜25 kDa).

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter. “Operably linked” in this context means two or more genetic elements, such as a polynucleotide coding sequence and a promoter, placed in relative positions that permit the proper biological functioning of the elements, such as the promoter directing transcription of the coding sequence. Other elements that may be present in an expression cassette include those that enhance transcription (e.g., enhancers) and terminate transcription (e.g., terminators), as well as those that confer certain binding affinity or antigenicity to the recombinant protein produced from the expression cassette.

The term “heterologous,” as used in the context of describing the relative location of two elements, refers to the two elements such as two polynucleotide sequences (e.g., a promoter and a polypeptide-encoding sequence) or polypeptide sequences (e.g., a first amino acid sequence, such as one set forth in SEQ ID NO:1, optionally with one or more mutations, and a second peptide sequence serving as a fusion partner with the first amino acid sequence) that are not found in the same relative position in nature. Thus, a “heterologous promoter” of a gene refers to a promoter that is not naturally operably linked to that gene. Similarly, a “heterologous polypeptide/amino acid sequence” or “heterologous polynucleotide” to an amino acid sequence or its encoding sequence is one derived from a non-vIL-6 origin or is derived from vIL-6 but not naturally connected to the first vIL-6-derived sequence (e.g., one set forth in SEQ ID NO:1) in the same fashion. The fusion of a vIL-6-derived amino acid sequence (or its coding sequence) with a heterologous polypeptide (or polynucleotide sequence) does not result in a longer polypeptide or polynucleotide sequence that can be found naturally in a vIL-6 sequence, including one that encompasses a signal sequence.

The term “inhibiting” or “inhibition,” as used herein, refers to any detectable negative effect on a target biological process, such as RNA/protein expression of a target gene, the biological activity of a target protein, protein-protein specific binding or interaction, cellular signal transduction, cell proliferation, presence/level of an organism especially a micro-organism, any measurable biomarker, bio-parameter, or symptom in a subject, and the like. Typically, an inhibition is reflected in a decrease of at least 10%, 20%, 30%, 40%, 50%, 60%, 70%, 80%, 90% or greater in the target process (e.g., a target ligand and receptor pair binding), or any one of the downstream parameters mentioned above, when compared to a control. “Inhibition” further includes a 100% reduction, i.e., a complete elimination, prevention, or abolition of a target biological process or signal or disease/symptom. The other relative terms such as “suppressing,” “suppression,” “reducing,” and “reduction” are used in a similar fashion in this disclosure to refer to decreases to different levels (e.g., at least 10%, 20%, 30%, 40%, 50%, 60%, 70%, 80%, 90% or greater decrease compared to a control level) up to complete elimination of a target biological process or signal or disease/symptom. On the other hand, terms such as “activate,” “activating,” “activation,” “increase,” “increasing,” “promote,” “promoting,” “enhance,” “enhancing,” or “enhancement” are used in this disclosure to encompass positive changes at different levels (e.g., at least about 5%, 10%, 20%, 30%, 40%, 50%, 60%, 70%, 80%, 90%, 100%, 200%, or greater such as 3, 5, 8, 10, 20-fold increase compared to a control level in a target process, signal, or symptom/disease incidence.

As used in this application, an “increase” or a “decrease” refers to a detectable positive or negative change in quantity from a comparison control, e.g., an established standard control (such as an average level of cancer cell proliferation rate). An increase is a positive change that is typically at least 10%, or at least 20%, or 50%, or 100%, and can be as high as at least 2-fold or at least 5-fold or even 10-fold of the control value. Similarly, a decrease is a negative change that is typically at least 10%, or at least 20%, 30%, or 50%, or even as high as at least 80% or 90% of the control value. Other terms indicating quantitative changes or differences from a comparative basis, such as “more,” “less,” “higher,” and “lower,” as well as terms indicating an action to cause such changes or differences, such as “increase,” “promote,” “enhance,” “decrease,” “inhibit,” and “suppress,” are used in this application in the same fashion as described above. In contrast, the term “substantially the same” or “substantially lack of change” indicates little to no change in quantity from the standard control value, typically within ±10% of the standard control, or within ±5%, 2%, or even less variation from the standard control.

As used herein, an “effective amount” or a “therapeutically effective amount” means the amount of a compound that, when administered to a subject or patient for treating a disorder, is sufficient to prevent, reduce the frequency of, or alleviate the symptoms of the disorder. The effective amount will vary depending on a variety of the factors, such as a particular compound used, the disease and its severity, the age, weight, and other factors of the subject to be treated. Amelioration of a symptom of a particular condition by administration of a pharmaceutical composition described herein refers to any lessening, whether permanent or temporary, that can be associated with the administration of the pharmaceutical composition. For example, the amount of a vIL-6 protein is considered therapeutically effective for treating a condition involving undesired/uncontrolled cellular proliferation or malignancy when treatment results in eliminated symptoms, delayed onset of symptoms, or reduced frequency or severity of symptoms including cancer progression, such as increase in tumor mass, metastasis, morbidity and mortality, etc.

As used herein, the term “treatment” or “treating” includes both therapeutic and preventative measures taken to address the presence of a disease or condition or the risk of developing such disease or condition at a later time. It encompasses therapeutic or preventive measures for alleviating ongoing symptoms, inhibiting or slowing disease progression, delaying of onset of symptoms, or eliminating or reducing side-effects caused by such disease or condition. A preventive measure in this context and its variations do not require 100% elimination of the occurrence of an event; rather, they refer to a suppression or reduction in the likelihood or severity of such occurrence or a delay in such occurrence.

A “subject,” or “subject in need of treatment,” as used herein, refers to an individual who seeks medical attention due to risk of, or actual sufference from, a condition involving an undesirable or abnormal thrombotic process or inflammatory response. The term subject can include both animals, especially mammals, and humans. Subjects or individuals in need of treatment include those that demonstrate symptoms of undesirable or inappropriate thrombosis such as thrombocytopenia and autoimmune response or are at risk of later developing these conditions and/or related symptoms.

The term “about” when used in reference to a given value denotes a range encompassing ±10% of the value.

A “pharmaceutically acceptable” or “pharmacologically acceptable” excipient is a substance that is not biologically harmful or otherwise undesirable, i.e., the excipient may be administered to an individual along with a bioactive agent without causing any undesirable biological effects. Neither would the excipient interact in a deleterious manner with any of the components of the composition in which it is contained.

The term “excipient” refers to any essentially accessory substance that may be present in the finished dosage form of the composition of this invention. For example, the term “excipient” includes vehicles, binders, disintegrants, fillers (diluents), lubricants, glidants (flow enhancers), compression aids, colors, sweeteners, preservatives, suspending/dispersing agents, film formers/coatings, flavors and printing inks.

The term “consisting essentially of,” when used in the context of describing a composition containing an active ingredient or multiple active ingredients, refer to the fact that the composition does not contain other ingredients possessing any similar or relevant biological activity of the active ingredient(s) or capable of enhancing or suppressing the activity, whereas one or more inactive ingredients such as physiological or pharmaceutically acceptable excipients may be present in the composition. For example, a composition consisting essentially of active agents (for instance, a vIL-6 polypeptide or a nucleic acid encoding a vIL-6 polypeptide) effective for enhancing or improving efficacy of immunotherapy for treating cancer in a subject is a composition that does not contain any other agents that may have any detectable positive or negative effect on the same target process (e.g., anti-cancer efficacy) or that may increase or decrease to any measurable extent of the disease severity or outcome among the receiving subjects.

### DETAILED DESCRIPTION OF THE INVENTION

### I. Introduction

Monocytes play an essential role in anti-tumor immunity through differentiating into dendritic cells and macrophages to acquire antigen-presenting functions¹. Although monocyte differentiation into dendritic cells or inflammatory M1 macrophages is desired for anti-tumor immunity, the tumor microenvironment is often associated with the development of suppressive M2 macrophages. The accumulation of M2 macrophages in tumors prevents mounting productive immune responses to kill tumors. Therefore, finding ways to block the generation of M2 macrophages and promote generation of dendritic cells and M1 macrophages is significant in cancer therapy.

Viral (v)IL-6 is a human (h)IL-6 homolog, encoded by Kaposi's sarcoma herpesvirus (KSHV), and is an inflammatory factor in patients with KSHV infections². Unlike hIL-6 that requires IL-6RA/gp130 heterodimeric receptor engagement to activate JAK/STAT pathway, vIL-6 can directly bind to the gp130 subunit of the IL-6 receptor to activate the JAK/STAT pathway by inducing STAT3 phosphorylation and acetylation, as well as the AKT pathway, to exhibit numerous biological effects^{3, 4, 5}. However, it is not fully understood how vIL-6 impacts the differentiation of monocytes.

This invention describes the discovery that a virally derived homolog of interleukin-6 promotes inflammatory responses but, unlike human IL-6, does not result in negative feedback regulation that then reduces the inflammatory response. The use of vIL-6 and this property of vIL-6 to increase inflammatory response may be desirable for such as cancer immunotherapy, by including but not limited to RNA- and viral vector-based delivery of vIL-6-encoding nucleic acids.

### II. Recombinant Expression of Polypeptides

### A. General Recombinant Technology

Basic texts disclosing general methods and techniques in the field of recombinant genetics include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1994).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22: 1859-1862 (1981), using an automated synthesizer, as described in Van Devanter et. al., Nucleic Acids Res. 12: 6159-6168 (1984). Purification of oligonucleotides is performed using any art-recognized strategy, e.g., native acrylamide gel electrophoresis or anion-exchange HPLC as described in Pearson & Reanier, J. Chrom. 255:137-149 (1983).

The sequence of a viral IL-6 gene, a polynucleotide encoding a polypeptide having the amino acid sequence SEQ ID NO:1 or its variants/mutants, and synthetic oligonucleotides can be verified after cloning or subcloning using, e.g., the chain termination method for sequencing double-stranded templates of Wallace et al., Gene 16: 21-26 (1981).

### B. Chemical Synthesis of Peptides

The amino acid sequence of vIL-6 protein and its nucleotide coding sequence are known and provided herein. A polypeptide comprising the full-length vIL-6 protein or a variant thereof including one or more point mutations thus can be chemically synthesized using conventional peptide synthesis or other protocols well-known in the art.

Polypeptides may be synthesized by solid-phase peptide synthesis methods using procedures similar to those described by Merrifield et al., J. Am. Chem. Soc., 85:2149-2156 (1963); Barany and Merrifield, Solid-Phase Peptide Synthesis, in The Peptides: Analysis, Synthesis, Biology Gross and Meienhofer (eds.), Academic Press, N.Y., vol. 2, pp. 3-284 (1980); and Stewart et al., Solid Phase Peptide Synthesis 2nd ed., Pierce Chem. Co., Rockford, Ill. (1984). During synthesis, N-α-protected amino acids having protected side chains are added stepwise to a growing polypeptide chain linked by its C-terminal and to a solid support, i.e., polystyrene beads. The peptides are synthesized by linking an amino group of an N-α-deprotected amino acid to an α-carboxy group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc, which is acid labile, and Fmoc, which is base labile.

Materials suitable for use as the solid support are well known to those of skill in the art and include, but are not limited to, the following: halomethyl resins, such as chloromethyl resin or bromomethyl resin; hydroxymethyl resins; phenol resins, such as 4-(α-[2,4-dimethoxyphenyl]-Fmoc-aminomethyl)phenoxy resin; tert-alkyloxycarbonyl-hydrazidated resins, and the like. Such resins are commercially available and their methods of preparation are known by those of ordinary skill in the art. Briefly, the C-terminal N-α-protected amino acid is first attached to the solid support. The N-α-protecting group is then removed. The deprotected α-amino group is coupled to the activated α-carboxylate group of the next N-α-protected amino acid. The process is repeated until the desired peptide is synthesized. The resulting peptides are then cleaved from the insoluble polymer support and the amino acid side chains deprotected. Longer peptides can be derived by condensation of protected peptide fragments. Details of appropriate chemistries, resins, protecting groups, protected amino acids and reagents are well known in the art and so are not discussed in detail herein (See, Atherton et al., Solid Phase Peptide Synthesis: A Practical Approach, IRL Press (1989), and Bodanszky, Peptide Chemistry, A Practical Textbook, 2nd Ed., Springer-Verlag (1993)).

### C. Recombinant Expression of Polypeptides

A vIL-6 protein of SEQ ID NO:1, its variant/mutant, or any fusion polypeptide comprising a wild-type vIL-6 protein or its variant/mutant can be produced using routine techniques in the field of recombinant genetics, relying on the polynucleotide sequences encoding the polypeptide disclosed herein.

To obtain high level expression of a nucleic acid encoding a desired polypeptide, one typically subclones a polynucleotide encoding the polypeptide into an expression vector that contains a strong promoter to direct transcription, a transcription/translation terminator and a ribosome binding site for translational initiation. Suitable bacterial promoters are well known in the art and described, e.g., in Sambrook and Russell, supra, and Ausubel et al., supra. Bacterial expression systems for expressing the polypeptide are available in, e.g., E. coli, Bacillus sp., Salmonella, and Caulobacter. Kits for such expression systems are commercially available. Eukaryotic expression systems for mammalian cells, yeast, and insect cells are well known in the art and are also commercially available. One exemplary eukaryotic expression vector is an adenoviral vector, an adeno-associated vector, or a retroviral vector.

Standard transfection methods can be used to produce bacterial, mammalian, yeast, insect, or plant cell lines that express large quantities of a recombinant polypeptide (e.g., a vIL-6 protein), which is then purified using standard techniques (see, e.g., Colley et al., J. Biol. Chem. 264: 17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132: 349-351 (1977); Clark-Curtiss & Curtiss, Methods in Enzymology 101: 347-362 (Wu et al., eds, 1983).

Any of the well-known procedures for introducing foreign nucleotide sequences into host cells may be used. These include the use of calcium phosphate transfection, polybrene, protoplast fusion, electroporation, liposomes, microinjection, plasma vectors, viral vectors and any of the other well-known methods for introducing cloned genomic DNA, cDNA, synthetic DNA, or other foreign genetic material into a host cell (see, e.g., Sambrook and Russell, supra). It is only necessary that the particular genetic engineering procedure used be capable of successfully introducing at least one gene into the host cell capable of expressing the recombinant polypeptide.

When a recombinant polypeptide, e.g., a vIL-6 protein, is expressed in host cells in satisfying quantity, its purification can follow the standard protein purification procedure including Solubility fractionation, size differential filtration, and column chromatography. These standard purification procedures are also suitable for purifying a vIL-6 protein (wild-type or mutant) obtained from chemical synthesis. The identity of the vIL-6 protein may be further verified by methods such as immunoassays (e.g., Western blot or ELISA) and mass spectrometry.

### III. Pharmaceutical Compositions and Administration

The present invention also provides pharmaceutical compositions comprising an effective amount of a vIL-6 protein for activating an immune response without triggering negative feedback mechanisms, therefore useful in the therapeutic applications designed for various diseases and conditions involving undesired and uncontrolled cellular proliferation. Pharmaceutical compositions of the invention are suitable for use in a variety of drug delivery systems. Suitable formulations for use in the present invention are found in Remington's Pharmaceutical Sciences, Mack Publishing Company, Philadelphia, PA, 17th ed. (1985). For a brief review of methods for drug delivery, see, Langer, Science 249: 1527-1533 (1990).

The pharmaceutical compositions of the present invention can be administered by various routes, e.g., oral, subcutaneous, transdermal, intramuscular, intravenous, or intraperitoneal. The routes of administering the pharmaceutical compositions include systemic or local delivery to a subject suffering from a condition exacerbated by undesired cell proliferation at daily doses of about 0.01-5000 mg, preferably 5-500 mg, of a vIL-6 polypeptide for a 70 kg adult human per day. The appropriate dose may be administered in a single daily dose or as divided doses presented at appropriate intervals, for example as two, three, four, or more sub-doses per day.

For preparing pharmaceutical compositions containing a vIL-6 polypeptide, inert and pharmaceutically acceptable carriers are used. The pharmaceutical carrier can be either solid or liquid. Solid form preparations include, for example, powders, tablets, dispersible granules, capsules, cachets, and suppositories. A solid carrier can be one or more substances that can also act as diluents, flavoring agents, solubilizers, lubricants, suspending agents, binders, or tablet disintegrating agents; it can also be an encapsulating material.

In powders, the carrier is generally a finely divided solid that is in a mixture with the finely divided active component, e.g., a vIL-6 polypeptide. In tablets, the active ingredient (the mutant polypeptide) is mixed with the carrier having the necessary binding properties in suitable proportions and compacted in the shape and size desired.

For preparing pharmaceutical compositions in the form of suppositories, a low-melting wax such as a mixture of fatty acid glycerides and cocoa butter is first melted and the active ingredient is dispersed therein by, for example, stirring. The molten homogeneous mixture is then poured into convenient-sized molds and allowed to cool and solidify.

Powders and tablets preferably contain between about 5% to about 70% by weight of the active ingredient. Suitable carriers include, for example, magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The pharmaceutical compositions can include the formulation of the active compound of a vIL-6 polypeptide with encapsulating material as a carrier providing a capsule in which the mutant (with or without other carriers) is surrounded by the carrier, such that the carrier is thus in association with the compound. In a similar manner, cachets can also be included. Tablets, powders, cachets, and capsules can be used as solid dosage forms suitable for oral administration.

Liquid pharmaceutical compositions include, for example, solutions suitable for oral or parenteral administration, suspensions, and emulsions suitable for oral administration. Sterile water solutions of the active component (e.g., a vIL-6 polypeptide) or sterile solutions of the active component in solvents comprising water, buffered water, saline, PBS, ethanol, or propylene glycol are examples of liquid compositions suitable for parenteral administration. The compositions may contain pharmaceutically acceptable auxiliary substances as required to approximate physiological conditions, such as pH adjusting and buffering agents, tonicity adjusting agents, wetting agents, detergents, and the like.

Sterile solutions can be prepared by dissolving the active component (e.g., a vIL-6 polypeptide) in the desired solvent system, and then passing the resulting solution through a membrane filter to sterilize it or, alternatively, by dissolving the sterile compound in a previously sterilized solvent under sterile conditions. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9, and most preferably from 7 to 8.

The pharmaceutical compositions containing the active ingredient (e.g., a vIL-6 protein) can be administered for prophylactic and/or therapeutic treatments. In therapeutic applications, compositions are administered to a patient already suffering from a condition that may be exacerbated by inappropriate or undesirable cell proliferation in an amount sufficient to prevent, cure, reverse, or at least partially slow or arrest the symptoms of the condition and its complications. An amount adequate to accomplish this is defined as a “therapeutically effective dose.” Amounts effective for this use will depend on the severity of the disease or condition and the weight and general state of the patient, but generally range from about 0.1 mg to about 2,000 mg of the mutant polypeptide per day for a 70 kg patient, with dosages of from about 5 mg to about 500 mg of the mutant polypeptide per day for a 70 kg patient being more commonly used.

In prophylactic applications, pharmaceutical compositions containing the active ingredient (e.g., a vIL-6 protein) are administered to a patient susceptible to or otherwise at risk of developing a disease or condition involving inappropriate or undesirable cellular proliferation in an amount sufficient to delay or prevent the onset of the symptoms. Such an amount is defined to be a “prophylactically effective dose.” In this use, the precise amounts of the inhibitor again depend on the patient's state of health and weight, but generally range from about 0.1 mg to about 2,000 mg of the mutant polypeptide for a 70 kg patient per day, more commonly from about 5 mg to about 500 mg for a 70 kg patient per day.

Single or multiple administrations of the compositions can be carried out with dose levels and pattern being selected by the treating physician. In any event, the pharmaceutical formulations should provide a quantity of a compound sufficient to effectively inducing an immune response without triggering a negative feedback mechanism mediated by vIL-6 in the patient, either therapeutically or prophylactically.

### IV. Therapeutic Applications Using Nucleic Acids

A variety of diseases and conditions due to or exacerbated by undesirable or excessive cell proliferation can be treated by therapeutic approaches that involve introducing into a cell a nucleic acid encoding a vIL-6 polypeptide such that the expression of the protein leads to an immune response with a reduced or abolished negative feedback. Those amenable to treatment by this approach include a broad spectrum of conditions and diseases of malignancy. For discussions on the application of gene therapy towards the treatment of genetic as well as acquired diseases, see, Miller Nature 357:455-460 (1992); and Mulligan Science 260:926-932 (1993).

### A. Vectors for Nucleic Acid Delivery

For delivery to a cell or organism, an inhibitory nucleic acid of the invention can be incorporated into a vector. Examples of vectors used for such purposes include expression plasmids capable of directing the expression of the vIL-6 polypeptides in the target cell. In other instances, the vector is a viral vector system wherein the polynucleotide is incorporated into a viral genome that is capable of transfecting the target cell. In a preferred embodiment, the inhibitory nucleic acid can be operably linked to expression and control sequences that can direct transcription of sequence in the desired target host cells. Thus, one can achieve reduced downstream effects medicated by vIL-6 under appropriate conditions in the target cell.

### B. Gene Delivery Systems

As used herein, “gene delivery system” refers to any means for the delivery of an inhibitory nucleic acid of the invention to a target cell. Viral vector systems useful in the introduction and expression of an inhibitory nucleic acid include, for example, naturally occurring or recombinant viral vector systems. Depending upon the particular application, suitable viral vectors include replication competent, replication deficient, and conditionally replicating viral vectors. For example, viral vectors can be derived from the genome of human or bovine adenoviruses, vaccinia virus, herpes virus, adeno-associated virus, minute virus of mice (MVM), HIV, sindbis virus, and retroviruses (including but not limited to Rous sarcoma virus), and MoMLV. Typically, the inhibitory nucleic acid is inserted into such vectors to allow packaging of the gene construct, typically with accompanying viral DNA, followed by infection of a sensitive host cell and expression of the gene of interest.

Similarly, viral envelopes used for packaging gene constructs that include the inhibitory nucleic acid can be modified by the addition of receptor ligands or antibodies specific for a receptor to permit receptor-mediated endocytosis into specific cells (see, e.g., WO 93/20221, WO 93/14188, and WO 94/06923).

Retroviral vectors may also be useful for introducing the inhibitory nucleic acid of the invention into target cells or organisms. Retroviral vectors are produced by genetically manipulating retroviruses. The viral genome of retroviruses is RNA. Upon infection, this genomic RNA is reverse transcribed into a DNA copy which is integrated into the chromosomal DNA of transduced cells with a high degree of stability and efficiency. The integrated DNA copy is referred to as a provirus and is inherited by daughter cells as is any other gene. The wild type retroviral genome and the proviral DNA have three genes: the gag, the pol and the env genes, which are flanked by two long terminal repeat (LTR) sequences. The gag gene encodes the internal structural (nucleocapsid) proteins; the pol gene encodes the RNA directed DNA polymerase (reverse transcriptase); and the env gene encodes viral envelope glycoproteins. The 5′ and 3′ LTRs serve to promote transcription and polyadenylation of virion RNAs. Adjacent to the 5′ LTR are sequences necessary for reverse transcription of the genome (the tRNA primer binding site) and for efficient encapsulation of viral RNA into particles (the Psi site) (see, Mulligan, In: Experimental Manipulation of Gene Expression, Inouye (ed), 155-173 (1983); Mann et al., Cell 33:153-159 (1983); Cone and Mulligan, Proceedings of the National Academy of Sciences, U.S.A., 81:6349-6353 (1984)).

The design of retroviral vectors is well-known to those of ordinary skill in the art. In brief, if the sequences necessary for encapsidation (or packaging of retroviral RNA into infectious virions) are missing from the viral genome, the result is a cis acting defect which prevents encapsidation of genomic RNA. However, the resulting mutant is still capable of directing the synthesis of all virion proteins. Retroviral genomes from which these sequences have been deleted, as well as cell lines containing the mutant genome stably integrated into the chromosome are well-known in the art and are used to construct retroviral vectors. Preparation of retroviral vectors and their uses are described in many publications including, e.g., European Patent Application EPA 0 178 220; U.S. Pat. No. 4,405,712; Gilboa Biotechniques 4:504-512 (1986); Mann et al., Cell 33:153-159 (1983); Cone and Mulligan Proc. Natl. Acad. Sci. USA 81:6349-6353 (1984); Eglitis et al. Biotechniques 6:608-614 (1988); Miller et al. Biotechniques 7:981-990 (1989); Miller (1992) supra; Mulligan (1993), supra; and WO 92/07943.

The retroviral vector particles are prepared by recombinantly inserting the desired inhibitory nucleic acid sequence into a retrovirus vector and packaging the vector with retroviral capsid proteins by use of a packaging cell line. The resultant retroviral vector particle is incapable of replication in the host cell but is capable of integrating into the host cell genome as a proviral sequence containing the desired nucleotide sequence. As a result, the patient is capable of producing, for example, the inhibitory nucleic acid, thus eliminating or reducing unwanted inflammatory conditions.

Packaging cell lines that are used to prepare the retroviral vector particles are typically recombinant mammalian tissue culture cell lines that produce the necessary viral structural proteins required for packaging, but which are incapable of producing infectious virions. The defective retroviral vectors that are used, on the other hand, lack these structural genes but encode the remaining proteins necessary for packaging. To prepare a packaging cell line, one can construct an infectious clone of a desired retrovirus in which the packaging site has been deleted. Cells comprising this construct will express all structural viral proteins, but the introduced DNA will be incapable of being packaged. Alternatively, packaging cell lines can be produced by transforming a cell line with one or more expression plasmids encoding the appropriate core and envelope proteins. In these cells, the gag, pol, and env genes can be derived from the same or different retroviruses.

A number of packaging cell lines suitable for the present invention are also available in the prior art. Examples of these cell lines include Crip, GPE86, PA317 and PG13 (see Miller et al., J. Virol. 65:2220-2224 (1991)). Examples of other packaging cell lines are described in Cone and Mulligan Proceedings of the National Academy of Sciences, USA, 81:6349-6353 (1984); Danos and Mulligan Proceedings of the National Academy of Sciences, USA, 85:6460-6464 (1988); Eglitis et al. (1988), supra; and Miller (1990), supra.

### C. Pharmaceutical Formulations

When used for pharmaceutical purposes, the inhibitory nucleic acid is generally formulated in a suitable buffer, which can be any pharmaceutically acceptable buffer, such as phosphate buffered saline or sodium phosphate/sodium sulfate, Tris buffer, glycine buffer, sterile water, and other buffers known to the ordinarily skilled artisan such as those described by Good et al. Biochemistry 5:467 (1966).

The compositions can further include a stabilizer, an enhancer, and/or other pharmaceutically acceptable carriers or vehicles. A pharmaceutically acceptable carrier can contain a physiologically acceptable compound that acts, for example, to stabilize the inhibitory nucleic acids of the invention and any associated vector. A physiologically acceptable compound can include, for example, carbohydrates, such as glucose, sucrose or dextrans, antioxidants, such as ascorbic acid or glutathione, chelating agents, low molecular weight proteins or other stabilizers or excipients. Other physiologically acceptable compounds include wetting agents, emulsifying agents, dispersing agents or preservatives, which are particularly useful for preventing the growth or action of microorganisms. Various preservatives are well known and include, for example, phenol and ascorbic acid. Examples of carriers, stabilizers or adjuvants can be found in Remington's Pharmaceutical Sciences, Mack Publishing Company, Philadelphia, PA, 17th ed. (1985).

### D. Administration of Formulations

The formulations containing a nucleic acid of interest (e.g., encoding vIL-6 protein) can be delivered to any tissue or organ using any delivery method known to the ordinarily skilled artisan. In some embodiments of the invention, the nucleic acid is formulated in mucosal, topical, and/or buccal formulations, particularly mucoadhesive gel and topical gel formulations. Exemplary permeation enhancing compositions, polymer matrices, and mucoadhesive gel preparations for transdermal delivery are disclosed in U.S. Pat. No. 5,346,701.

The formulations containing the inhibitory nucleic acid are typically administered to a cell. The cell can be provided as part of a tissue or as an isolated cell, such as in tissue culture. The cell can be provided in vivo, ex vivo, or in vitro.

The formulations can be introduced into the tissue of interest in vivo or ex vivo by a variety of methods. In some embodiments of the invention, the inhibitory nucleic acid is introduced into cells by such methods as microinjection, calcium phosphate precipitation, liposome fusion, ultrasound, electroporation, or biolistics. In further embodiments, the nucleic acid is taken up directly by the tissue of interest.

In some embodiments of the invention, the inhibitory nucleic acid is administered ex vivo to cells or tissues explanted from a patient, then returned to the patient. Examples of ex vivo administration of therapeutic gene constructs include Nolta et al., Proc Natl. Acad. Sci. USA 93(6):2414-9 (1996); Koc et al., Seminars in Oncology 23(1):46-65 (1996); Raper et al., Annals of Surgery 223(2):116-26 (1996); Dalesandro et al., J. Thorac. Cardi. Surg., 11(2):416-22 (1996); and Makarov et al., Proc. Natl. Acad. Sci. USA 93(1):402-6 (1996).

Effective dosage of the formulations will vary depending on many different factors, including means of administration, target site, physiological state of the patient, and other medicines administered. Thus, treatment dosages will need to be titrated to optimize safety and efficacy. In determining the effective amount of the vector to be administered, the physician should evaluate the particular nucleic acid used, the disease state being diagnosed; the age, weight, and overall condition of the patient, circulating plasma levels, vector toxicities, progression of the disease, and the production of anti-vector antibodies. The size of the dose also will be determined by the existence, nature, and extent of any adverse side-effects that accompany the administration of a particular vector. To practice the present invention, doses ranging from about 10 ng-1 g, 100 ng-100 mg, 1 μg-10 mg, or 30-300 μg inhibitory nucleic acid per patient are typical. Doses generally range between about 0.01 and about 50 mg per kilogram of body weight, preferably between about 0.1 and about 5 mg/kg of body weight or about 10⁸-10¹⁰ or 10¹² viral particles per injection. In general, the dose equivalent of a naked nucleic acid from a vector is from about 1 μg-100 μg for a typical 70 kg patient, and doses of vectors which include a retroviral particle are calculated to yield an equivalent amount of an inhibitory nucleic acid.

### V. Anti-Cancer Therapeutic Agents

Given the activity of vIL-6 in triggering an anti-cancer immune response without triggering a negative feedback mechanism, previously known therapeutic agent or agents with anti-cancer efficacy may be used in combination with a vIL-6 protein or nucleic acid as described herein during the practice of the present invention for the purpose of effectively treating cancer. In such applications, one or more of these previously known effective anti-cancer therapeutic agents can be administered to patients concurrently with an effective amount of the vIL-6 formulation either together in a single composition or separately in two or more different compositions. They may be used in combination with the active agent of the present invention (e.g., the vIL-6 protein or nucleic acid) to suppress cancer growth, inhibit cancer metastasis, and facilitate remission from the disease.

For example, various chemotherapeutic agents are known to be effective for use to treat various cancers. As used herein, a “chemotherapeutic agent” encompasses any chemical compound exhibiting suppressive effect against cancer cells, thus useful in the treatment of cancer. Classes of chemotherapeutic agents include, but are not limited to: alkylating agents, antimetabolites, kinase inhibitors, spindle poison plant alkaloids, cytoxic/antitumor antibiotics, topoisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists, anti-androgens, aromatase inhibitors, EGFR inhibitors, VEGF inhibitors, and anti-sense oligonucleotides that inhibit expression of genes implicated in abnormal cell proliferation or tumor growth. Chemotherapeutic agents useful in the treatment methods disclosed herein also include cytostatic and/or cytotoxic agents.

Exemplary anti-cancer therapeutic agents include alkylating agents such as altretamine, bendamustine, busulfan, carboquone, carmustine, chlorambucil, chlormethine, chlorozotocin, cyclophosphamide, dacarbazine, fotemustine, ifosfamide, lomustine, melphalan, melphalan flufenamide, mitobronitol, nimustine, nitrosoureas, pipobroman, ranimustine, semustine, streptozotocin, temozolomide, thiotepa, treosulfan, triaziquone, triethylenemelamine, trofosfamide, and uramustine; anthracyclines such as aclarubicin, daunorubicin, doxorubicin, epirubicin, idarubicin, mitoxantrone, pirarubicin, valrubicin, and zorubicin; cytoskeletal disruptors (taxanes) such as abraxane, cabazitaxel, docetaxel, larotaxel, paclitaxel, taxotere, and tesetaxel; epothilones such as ixabepilone; histone deacetylase inhibitors such as vorinostat, romidepsin, and inhibitors of topoisomerase I such as belotecan, camptothecin, exatecan, gimatecan, irinotecan, and topotecan; inhibitors of topoisomerase II such as etoposide, teniposide, and tafluposide; kinase inhibitors such as bortezomib, erlotinib, gefitinib, imatinib, vemurafenib, and vismodegib; nucleotide analogs and precursor analogs such as azacitidine, azathioprine, capecitabine, cytarabine, doxifluridine, fluorouracil, gemcitabine, hydroxyurea, mercaptopurine, methotrexate, and tioguanine (formerly thioguanine); peptide antibiotics such as actinomycin and bleomycin; platinum-based agents such as carboplatin, cisplatin, dicycloplatin, oxaliplatin, nedaplatin, and satraplatin; retinoids such as alitretinoin, bexarotene, and tretinoin; and vinca alkaloids and derivatives such as vinblastine, vincristine, vindesine, and vinorelbine.

Immunotherapeutic approaches useful for cancer treatment include (1) active immunotherapy, which directs the immune system to specifically target the cancer cells, e.g., targeted antibody therapy and cell-based immunotherapy such as CAR T cell therapy; and (2) passive immunotherapy, e.g., using checkpoint inhibitors and cytokines to stimulate the immune system without specifically targeting cancer cells. Various monoclonal antibodies are used in targeted antibody therapy. Examples of such antibodies and their conjugates include adotrastuzumab (HER2), alemtuzumab (CD52), bevacizumab (VEGF), brentuximab (CD30), capromab (PSMA), cetuximab (EGFR), elotuzumab (SLAMF7), ibritumomab (CD20), necitumumab (EGFR), obinutumab (CD20), ofatumumab (CD20), olaratumab (PDGFRA), panitumumab (EGFR), pertuzumab (HER2), ramucirumab (VEGFR2), rituximab (CD-20), trastuzumab (HER-2), inotuzumab-ozogamicin (CD22), gemtuzumab-ozogamicin (CD33), and bevacizumab-awwb (VEGF). Currently approved checkpoint inhibitors target molecules CTLA4, PD-1, and PD-L1, including ipilimumab (CTLA4), nivolumab, pembrolizumab, cemiplimab, spartalizumab (PD-1), atezolizumab, avelumab, and durvalumab (PD-L1). Cytokines for use in the treatment of cancer and associated conditions include granulocyte colony-stimulating factor (G-CSF), granulocyte macrophage colony-stimulating factor (GM-CSF), interleukin-2 (IL-2), and interleukin-11 (IL-11).

### VI. Kits

The invention also provides kits for preventing or treating a condition involving undesirable cellular proliferation including various types of cancer by administering a vIL-6 protein or nucleic acid according to the method of the present invention. The kits typically include a first container that contains a pharmaceutical composition having an effective amount of a vIL-6 protein, its variant, or a nucleic acid encoding the protein or variant, optionally with a second container containing an anti-cancer agent, which may belong to any of the following 3 categories: (A) chemotherapeutic drugs, e.g., drugs capable of killing or suppressing cells that are actively undergoing proliferation; (B) immunotherapeutic agents, e.g., monoclonal antibodies for targeted antibody therapy, checkpoint inhibitors, and cytokines; and (C) cell-based therapeutic agents, e.g., those used in CAR T cell therapy or dendritic cell therapy.

In some cases, the kits will also include informational material containing instructions on how to dispense the pharmaceutical composition, including description of the type of patients who may be treated (e.g., a person suffering from a condition involving undesirable/uncontrolled cellular proliferation such as any type of malignancy), the schedule (e.g., dose and frequency of administration) and route of administration, and the like.

### EXAMPLES

The following examples are provided by way of illustration only and not by way of limitation. Those of skill in the art will readily recognize a variety of non-critical parameters that could be changed or modified to yield essentially the same or similar results.

### Example 1

Recombinant vIL-6 Generation

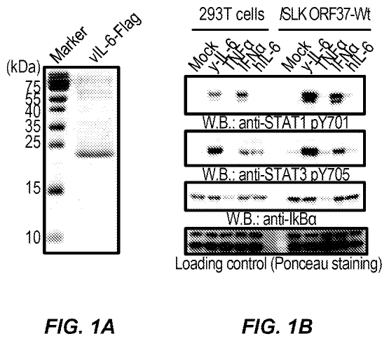

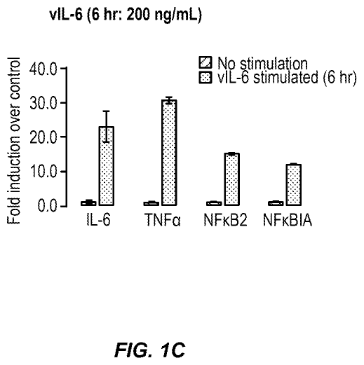

To test the effect of vIL-6 on monocyte differentiation, recombinant KSHV vIL-6 protein was expressed using a recombinant baculovirus system in Sf9 cells and purified with Flag agarose beads (FIG. 1A). To confirm the biological activity, two cell lines were treated with vIL-6 as well as control cytokines, IFNα, TNFα, and human IL-6 (hIL-6), to evaluate STAT1 and 3 activations. As shown in FIG. 1B, purified vIL-6 showed strong STAT1 activation in addition to STAT3 activation as previously reported^{3, 4}. Also, when monocytic THP-1 cell lines were treated with vIL-6 (200 ng/ml 6 hours), inflammatory gene expression (IL-6, TNFα, NFkB2, and NFkB1A) was increased over 10-fold compared to cells without stimulation. These results confirmed that recombinant vIL-6 has biological activity consistent with previous reports.

### VIL-6 Induces M1-Like Phenotype in Human Monocytes

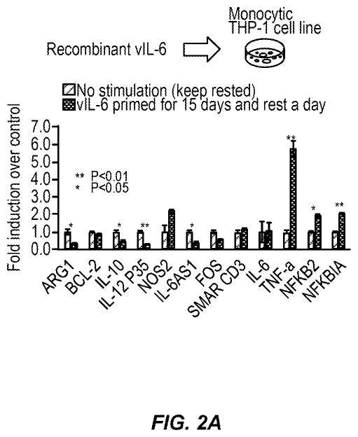

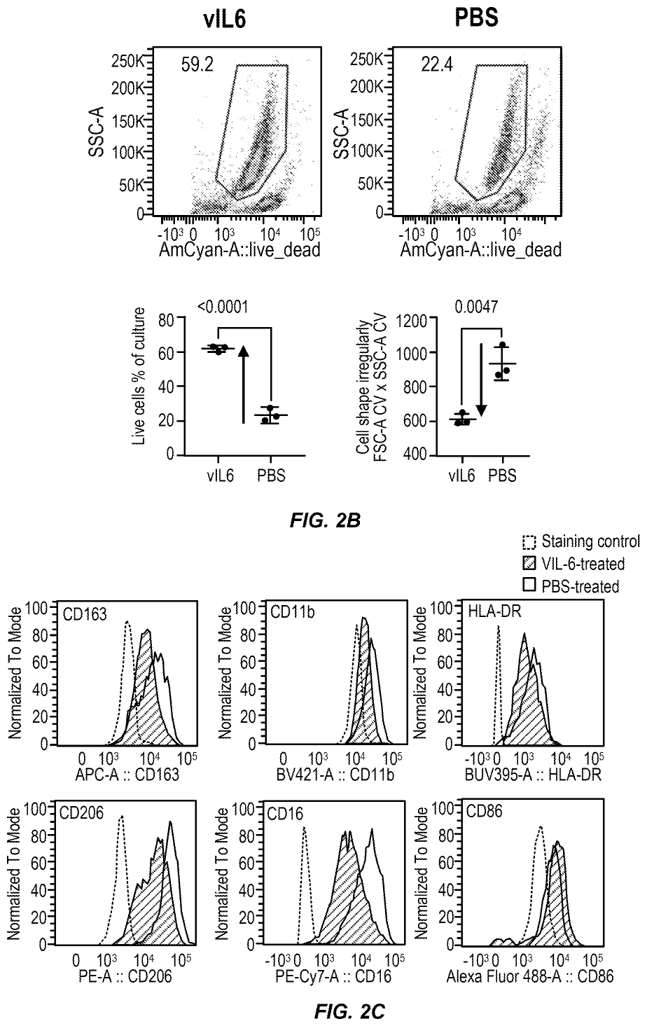

Next, the long-term effect of vIL-6 on monocyte differentiation was tested. First, monocytic human cell line THP-1 cells were cultured without or with vIL-6 (200 ng/ml) every day for 2 weeks followed by 24 hrs resting (no vIL-6), and gene expression was measured by qRT-PCR with beta-actin for normalization. As shown in FIG. 2A, genes associated with inflammatory responses including TNFa, NFkB, and NOS2, were significantly upregulated whereas IL-10 and ARG1, markers for suppressive M2 macrophages, were reduced in vIL-6-treated THP-1 cells.

The effect of vIL-6 on monocyte differentiation was also examined. Monocytes were isolated from peripheral blood mononuclear cells (PBMC) by negative selection of magnetic beads, and then cultured (5×10⁶/ml, 400 uL in 48 well plate, 10% FBS RPMI1640) without (PBS control) or with vIL-6 (100 ng/ml) for 1-2 weeks to allow differentiation into macrophages. As shown in FIG. 2B, viability of monocyte cultures was significantly higher in the presence of vIL-6 compared to PBS control. Also, vIL-6-treated cells showed uniformly round shape, indicative of differentiation of M1 type macrophages. The latter was evident from reduced cell irregularity based on FSC and SSC values compared to PBS control cultures. Furthermore, flow cytometry analysis of monocytes one week after vIL-6 treatment demonstrated that M2 markers (CD163, CD206, CD11b, CD16) were reduced without affecting on M1 markers (HLA-DR, CD86) (FIG. 2C). These results indicate that vIL-6 treatment shifts macrophage towards M1 phenotype by reducing M2 phenotype, which is favorable for anti-tumor immunity.

### VIL-6 Promotes Dendritic Cell Differentiation and Reduces Macrophage Differentiation

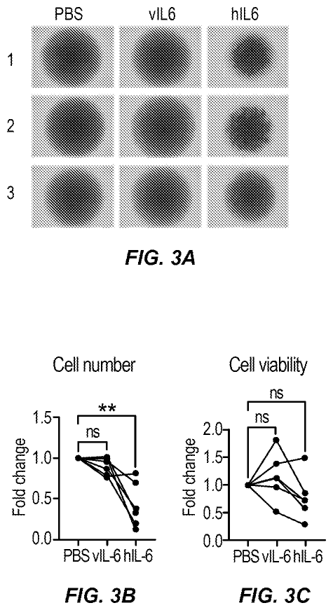

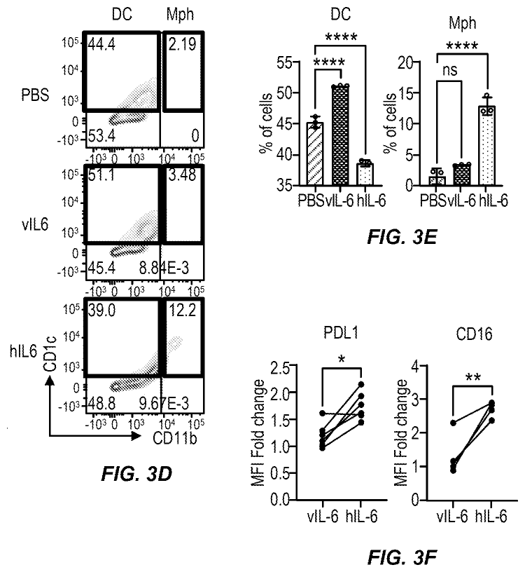

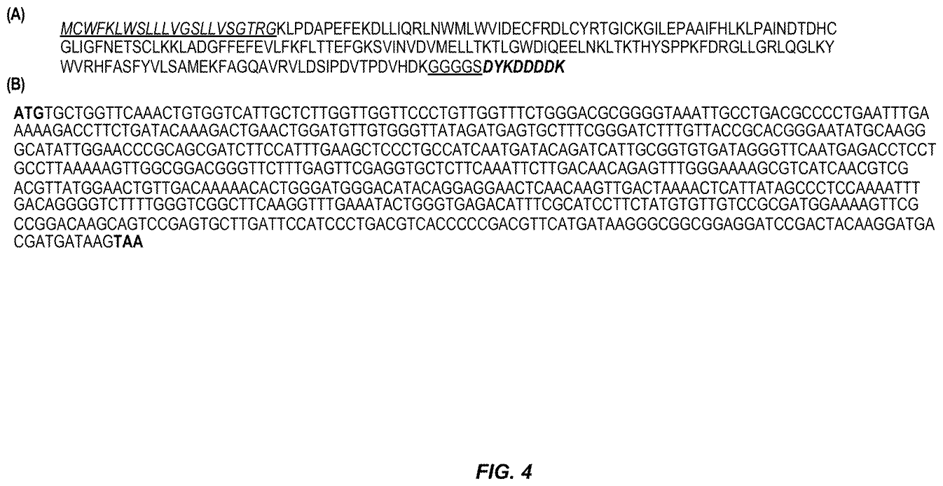

Dendritic cells are essential antigen-presenting cells for cytotoxic response in anti-tumor immunity. The effect of vIL-6 on monocyte differentiation into dendritic cells was examined. Monocytes were isolated from PBMC of 6 healthy donors by negative selection of magnetic beads, and then cultured in the presence of GM-CSF and IL-4 (2×10⁶/ml, 200 uL in 96 U-bottom well plate, 10% FBS RPMI1640, 50 ng/ml GM-CSF, 50 ng/ml IL-4) with PBS (control), vIL-6 (100 ng/ml), or hIL-6 (100 ng/ml) for 1-2 weeks to allow dendritic cell differentiation. It has previously been shown that hIL-6 stimulation results in negative feedback mechanisms to regulate inflammatory response⁶. Consistent with this, 2 weeks of hIL-6 treatment of monocytes results in smaller cellular mass volumes compared to PBS control treatment (FIG. 3A). Unlike hIL-6, however, vIL-6 did not impair cell proliferation and survival (FIGS. 3B and C). Thus, vIL-6 stimulation does not induce negative regulatory feedback to dampen inflammatory response, the property favorable for anti-tumor immunity.

Also, hIL-6 has been shown to switch the differentiation of monocytes from dendritic cells to monocytes⁷. Consistent with this, we observed that hIL-6 treatment significantly reduced the frequency of dendritic cells and enhanced the frequency of macrophages in the culture (FIGS. 3D and E), which is not ideal for anti-tumor immunity. Unlike hIL-6, however, vIL-6 treatment enhanced dendritic cell frequency without enhancing macrophage frequency (FIGS. 3D and E). Moreover, the expression of inhibitory markers PD-L1 and CD16 was significantly reduced in vIL-6-treated samples compared to hIL-6-treated samples (FIG. 3F). These properties of vIL-6 are ideal for anti-tumor immunity.

Collectively, these results demonstrate that vIL-6 can be used to prepare dendritic cells with better functionality and recovery ideal for dendritic cell transfer therapy as well as to boost anti-tumor immunity in combination with currently available cancer therapies, for example, those named in FIG. 3: therapies employing one or more checkpoint inhibitors (e.g., anti-CTLA-4, anti-PD-1, anti-PD-L1, anti-LAG-3, and the like); cancer vaccine therapies; and T cell transfer therapy such as CAR-T cell therapy.

### Example 2

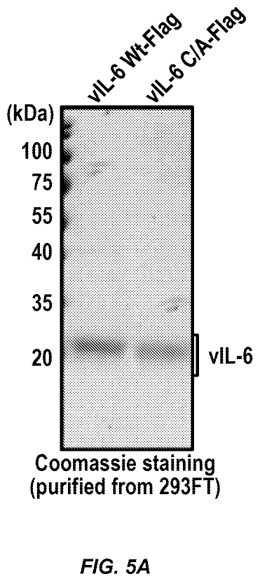

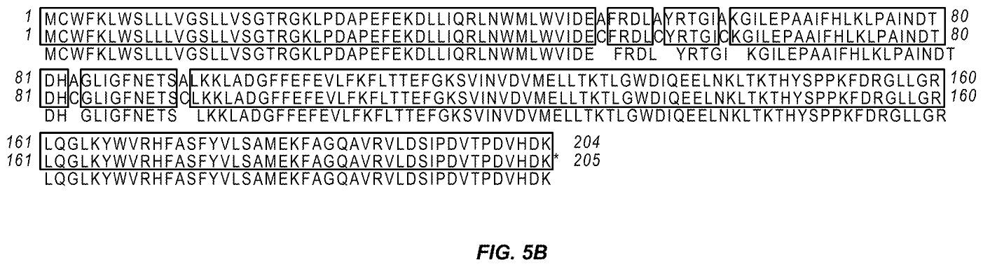

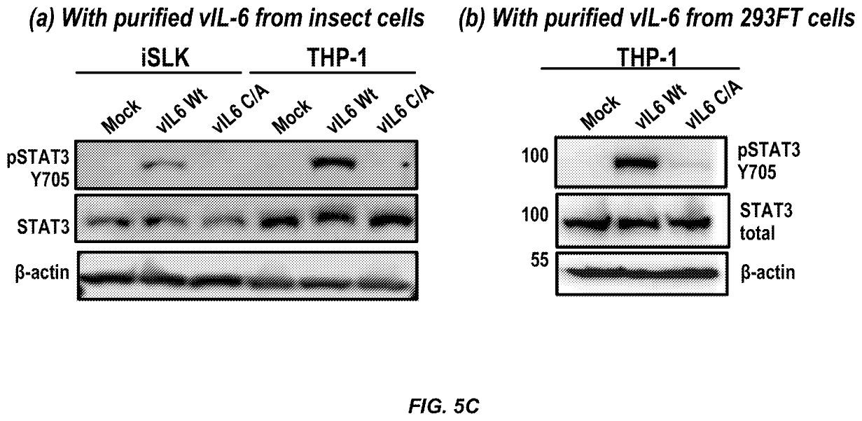

Recombinant vIL-6 production in human cell line for cGMP production. For future cGMP production purposes, recombinant KSHV wild type vIL-6 protein (vIL-6-Wt-Flag) along with a loss-of-function vIL-6 C/A mutant protein (vIL-6-C/A-Flag) as a control for signal transduction profiling were produced by human 293FT cell lines and purified with Flag agarose beads. The agarose gel revealed a single band corresponding the correct molecular weight (FIG. 5A).

To confirm the biological activity, human monocytic cell line THP-1 cells were treated with vIL-6 as well as a loss-of-function vIL-6 C/A mutant protein to evaluate STAT3 activation. The assays were run with wild type vIL-6 and mutant vIL-6 C/A previously produced from insect Sf9 cell lines (a) and those produced from human 293 FT cell lines (b). As shown in FIG. 5B, in both preparations (a and b), purified wild type vIL-6 showed strong STAT3 activation as previously reported,^{3, 4} whereas vIL-6 C/A mutant protein did not. These results confirmed that recombinant vIL-6 produced in human cell lines has biological activity consistent with previous reports. Collectively, these data demonstrate the feasibility of vIL-6 production in human cell lines for future cGMP production.

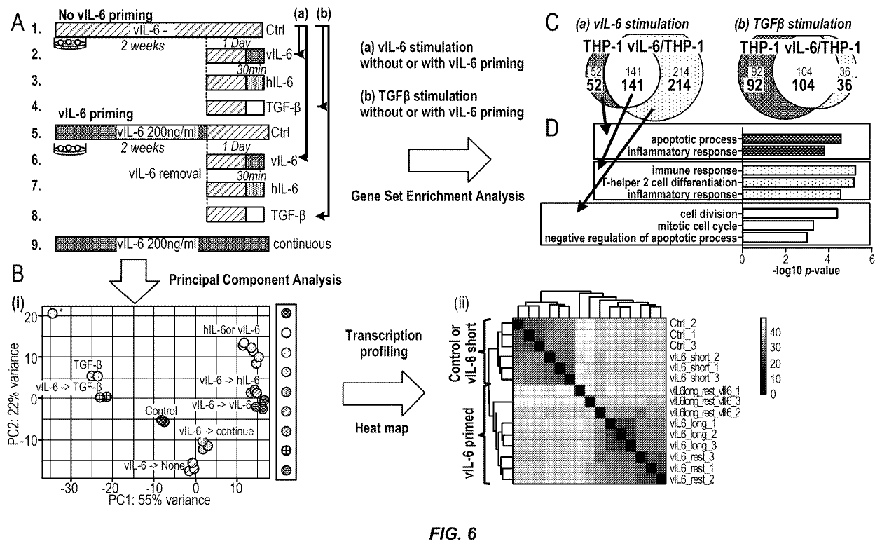

Gene expression analysis of human THP-1 cell line after vIL-6 stimulation revealed the impact of vIL-6 in monocytic cells to promote proliferation. To study a long-term impact of vIL-6, human monocytic cell line THP-1 was treated in various conditions with vIL-6 and TGFβ, as an irrelevant stimulus control, as illustrated in FIG. 6A (Experiment 1-9). Briefly, THP-1 cells were primed without (Experiment 1-4) or with (Experiment 5-8) vIL-6 for 2 weeks, rested in fresh medium without vIL-6 for 1 day, and treated without (Experiment 1 and 5) or with vIL-6 (Experiment 2 and 6) or TGFβ (Experiment 4 and 8) for 30 min before RNA-seq analysis. Human IL-6 treatment was also included (Experiment 3 and 7) along with continuous vIL-6 treatment throughout (Experiment 9) as controls. Various types of transcription profiling were then conducted (FIGS. 6B, C, and D).

As shown in FIG. 6B (i), Principal Component Analysis of Experiment 1-9 in (A) demonstrates that THP-1 cell groups either long vIL-6-primed or vIL-6 short-treated clearly formed separated clusters, indicating that the long-term vIL-6 treatment changes the gene expression landscape of monocytic cells. This is also visualized by heatmap in FIG. 6B (ii) that clearly separates transcriptional profiles of THP-1 cells with long vIL-6-primed from those with vIL-6 short-treated. Furthermore, in FIG. 6C, Venn diagrams for THP-1 cells that were primed without (THP-1 in red circle) or with vIL-6 (vIL-6/THP-1 in green circle) followed by stimulation with (a) vIL-6 or (b) TGFβ, as a control, show the numbers of genes differentially or commonly expressed in each group. By comparing THP-1 cells stimulated with vIL-6 (a) and a control cytokine TGFβ (b), genes differentially expressed only in vIL-6-primed THP-1 cells were substantially higher upon restimulation with vIL-6 (214 genes) than that with TGFβ (36 genes), indicating that vIL-6 priming upregulates a group of genes in THP-1 cells. Following Gene Set Enrichment Analysis (GSEA) in FIG. 6D revealed that a group of genes that are differentially expressed only in vIL-6 primed THP-1 cells (a green circle in C (a)) are enriched with genes associated with cell proliferation and negative regulation of apoptosis (shown in green). Collectively, gene expression profiling indicates that a long-term vIL-6 treatment induces gene expression changes in monocytic cells to promote cell proliferation.

### REFERENCES

[1] Duan, Z., and Luo, Y. (2021) Targeting macrophages in cancer immunotherapy, Signal Transduction and Targeted Therapy 6, 127.
[2] Aoki, Y., et al. (2001) Detection of viral interleukin-6 in Kaposi sarcoma-associated herpesvirus-linked disorders, Blood 97, 2173-2176.
[3] Molden, J., et al. (1997) A Kaposi's sarcoma-associated herpesvirus-encoded cytokine homolog (vIL-6) activates signaling through the shared gp130 receptor subunit, J Biol Chem 272, 19625-19631.
[4] Hu, F., and Nicholas, J. (2006) Signal transduction by human herpesvirus 8 viral interleukin-6 (vIL-6) is modulated by the nonsignaling gp80 subunit of the IL-6 receptor complex and is distinct from signaling induced by human IL-6, J Virol 80, 10874-10878.
[5] Zhu, X., et al. (2014) Synergy between Kaposi's sarcoma-associated herpesvirus (KSHV) vIL-6 and HIV-1 Nef protein in promotion of angiogenesis and oncogenesis: role of the AKT signaling pathway, Oncogene 33, 1986-1996.
[6] Yasukawa, H., et al. (2003) IL-6 induces an anti-inflammatory response in the absence of SOCS3 in macrophages, Nat Immunol 6, 551-556.
[7] Chomarat, P., et al. (2000) IL-6 switches the differentiation of monocytes from dendritic cells to macrophages, Nat Immunol 1, 510-514.

All patents, patent applications, and other publications, including GenBank Accession Numbers, cited in this application are incorporated by reference in the entirety for all purposes.

1-9. (canceled)

10. A composition comprising (1) vIL-6; (2) an anti-cancer therapeutic agent; and (3) a physiologically acceptable excipient.

11. The composition of claim 10, wherein the vIL-6 is vIL-6 protein.

12. The composition of claim 10, wherein the vIL-6 is a nucleic acid comprising a polynucleotide sequence encoding vIL-6 protein.

13. The composition of claim 12, wherein the nucleic acid is DNA.

14. The composition of claim 12, wherein the nucleic acid is RNA.

15. The composition of claim 12, the nucleic acid is a viral vector.

16. The composition of claim 15, wherein the viral vector is an adenoviral vector or adenovirus-associated viral vector.

17. A kit for treating cancer, comprising a first container containing a first composition comprising vIL6 and a second container containing a second composition comprising an anti-cancer therapeutic agent.

18. The kit of claim 17, wherein the vIL-6 is vIL-6 protein.

19. The kit of claim 17, wherein the vIL-6 is a nucleic acid comprising a polynucleotide sequence encoding vIL-6 protein.

20-23. (canceled)

24. A method for enhancing efficacy of dendritic cell transfer therapy, comprising supplementation of a culture medium formulated for dendritic cell production from monocytes with an effective amount of vIL6 prior to administering the dendritic cells derived thereof to a patient who has been diagnosed with a cancer.

25. The method of claim 24, wherein the vIL-6 is vIL-6 protein.

26. The method of claim 24, wherein the vIL-6 is a nucleic acid comprising a polynucleotide sequence encoding vIL-6 protein.

27. The method of claim 24, further comprising, prior to the incubating, obtaining the dendritic cells from the patient or from induced monocytes.

28-31. (canceled)

32. A method for treating cancer in a patient in need thereof, comprising administering to the patient an effective amount of the composition of claim 10.

33. The method of claim 32, wherein the vIL-6 is vIL-6 protein.

34. The method of claim 32, wherein the vIL-6 is a nucleic acid comprising a polynucleotide sequence encoding vIL-6 protein.


SEQUENCE LISTING (9 sequences)

SEQ ID NO: 1 - protein, 182 aa, synthetic construct
  REGION 1..182: note = Description of sequence: exemplary vIL-6 protein
KLPDAPEFEKDLLIQRLNWMLWVIDECFRDLCYRTGICKGILEPAAIFHLKLPAINDTDHCGLIGFNETSCLKKLADGFFEFEVLFKFLTTEFGKSVINVDVMELLTKTLGWDIQEELNKLTKTHYSPPKFDRGLLGRLQGLKYWVRHFASFYVLSAMEKFAGQAVRVLDSIPDVTPDVHDK

SEQ ID NO: 2 - protein, 214 aa, synthetic construct
  REGION 1..214: note = Description of sequence: vIL-6 amino acid sequence
MCWFKLWSLLLVGSLLVSGTRGKLPDAPEFEKDLLIQRLNWMLWVIDECFRDLCYRTGICKGILEPAAIFHLKLPAINDTDHCGLIGFNETSCLKKLADGFFEFEVLFKFLTTEGFKSVINVDMELLTXTLGWDIQEELNKLTKTHYSPPKFDRGLLGRLQGLKYWVRHFASFYVLSAMEKFAGQAVRVLDSIPDVTPVHDKGGGGSDYDDDDK

SEQ ID NO: 3 - DNA, 655 bp, synthetic construct
  misc_feature 1..655: note = Description of sequence: KSHV K2 open reading frame
  source 1..655: mol_type = other DNA
atgtgctggttcaaactgtggtcattgctcttggttggttccctgctggtttctgggacgcggggtaaattgcctgacgcccctgaatttgaaaaagaccttctgatacaaagactcaattggatgttgtgggttatagatgagtgctttcgggatctttgttaccgcacgggaatatgcaagggcatattggaacccgcagcgatcttccatttgaagctccctgccatcaatgatacagatcattgcggtctcatagggttcaatgagacctcctgccttaaaaagttggcggacgggttctttgagttcgaggtgctcttcaaattcttgacaacagagtttgggaaaagcgtcatcaacgtcgacgttatggaactgttgacaaaaacactgggatgggacatacaggaggaactcaacaagttgactaaaactcattatagccctccaaaatttgacaggggtcttttgggtcggcttcaaggatttgaaatactgggtgagacatttcgcatccttctatgtgttgtccgcgatggaaaagttcgccggacaagcagtccgagtgcttgattccatccctgacgtcacccccgacgttcatgataagggcggcggaggatccgactacaaggatgacgatgataagtaa

SEQ ID NO: 4 - protein, 5 aa, synthetic construct
  REGION 1..5: note = Description of sequence: Flexible linker
GGGGS

SEQ ID NO: 5 - protein, 8 aa, synthetic construct
  REGION 1..8: note = Description of sequence: Flag Tag
DYKDDDDK

SEQ ID NO: 6 - protein, 22 aa, synthetic construct
  REGION 1..22: note = Description of sequence: Signal sequence
MCWFKLWSLLLVGSLLVSGTRG

SEQ ID NO: 7 - protein, 202 aa, synthetic construct
  REGION 1..202: note = Description of sequence: comparison of vIL-6-Wt
MCWFKLWSLLLVGSLLVSGTRGKLPDAPEFEKDLLIQRLNWMLWVIDEAFRDLAYRTGIAKGILEPAAIFHLKLPAINDTDHAGLIGFNETSALKKLADGFFEFEVLFKFLTTEGFKSVINVDMELLTXTLGWDIQEELNKLTKTHYSPPKFDRGLLGRLQGLKYWVRHFASFYVLSAMEKFAGQAVRVLDSIPDVTPVHDK

SEQ ID NO: 8 - protein, 202 aa, synthetic construct
  REGION 1..202: note = Description of sequence: vIL-6 C/A mutant
MCWFKLWSLLLVGSLLVSGTRGKLPDAPEFEKDLLIQRLNWMLWVIDECFRDLCYRTGICKGILEPAAIFHLKLPAINDTDHCGLIGFNETSCLKKLADGFFEFEVLFKFLTTEGFKSVINVDMELLTXTLGWDIQEELNKLTKTHYSPPKFDRGLLGRLQGLKYWVRHFASFYVLSAMEKFAGQAVRVLDSIPDVTPVHDK

SEQ ID NO: 9 - protein, 202 aa, synthetic construct
  REGION 1..202: note = Description of sequence: consensus of vIL-6-Wt and vIL-6 C/A mutant
  VARIANT 49: note = X is point of inconsistency in the consensus sequence
  VARIANT 54: note = X is point of inconsistency in the consensus sequence
  VARIANT 60: note = X is point of inconsistency in the consensus sequence
  VARIANT 83: note = X is point of inconsistency in the consensus sequence
  VARIANT 93: note = X is point of inconsistency in the consensus sequence
MCWFKLWSLLLVGSLLVSGTRGKLPDAPEFEKDLLIQRLNWMLWVIDEXFRDLXYRTGIXKGILEPAAIFHLKLPAINDTDHXGLIGFNETSXLKKLADGFFEFEVLFKFLTTEGFKSVINVDMELLTXTLGWDIQEELNKLTKTHYSPPKFDRGLLGRLQGLKYWVRHFASFYVLSAMEKFAGQAVRVLDSIPDVTPVHDK